(19) United States
(12) Patent Application Publication
(10) Pub. No.: US 2008/0299062 A1
(43) Pub. Date: Dec. 4, 2008

(54) Use of Oxocarboxylic Acids-Containing Combinations for Deodorization

(76) Inventors: Thomas Kripp, Fraenkisch-Crumbach (DE); Carolin Harpe, Lorsch (DE); Walter Keller, Ober-Ramstadt (DE); Karl-Heinz Kischka, Darmstadt (DE)

Correspondence Address: MICHAEL J. STRIKER, 103 EAST NECK ROAD, HUNTINGTON, NY, 11743, US

(21) Appl. No.: 11/571,774

(22) PCT Filed: Jul. 11, 2005
(86) PCT No.: PCT/EP05/07485

§ 371 Date: Jan. 8, 2007

(30) Foreign Application Priority Data
Jul. 9, 2004 (DE) 10 2004 033 206.1

(51) Int. Cl. A61K 8/30; A61Q 15/00; A61Q 5/00

(52) U.S. Cl. 424/65; 424/70.5

(57) ABSTRACT

Described is the use of a combination of an oxocarboxylic acid with metal ions, organic bases or polymers substantive for keratin material for combating, suppressing or eliminating odors of the hair of skin. Preferred oxocarboxylic acids are those of general formula R—C(═O)—A—CO₂H wherein R stands for hydrogen or a monovalent organic group and A strands for a divalent organic group or for a single bond. A preferred combination is zinc levulinate which can be used, in particular, for combating the unpleasant odors of sulfur compounds, for example after permanent wave treatments.

The present invention has for an object the use of oxocarboxylic acids in combination with metal ions, organic bases or substantive polymers for deodorization, namely for combating, suppressing or eliminating odors of the hair or skin, and the corresponding cosmetic agents and methods of hair treatment.

Since the introduction of chemical hair reshaping (cold permanent waves) in the year 1940, numerous agents have been tested worldwide which were intended to cause transient softening of the hair by means of reduction processes. Although a large number of reducing agents belonging to the most varied classes of compounds are known, eventually only a few were accepted for permanent wave and hair-smoothing treatments. The common feature of all these compounds is that they contain a thiol group as the reducing moiety. Neither hydrides nor reductones nor other inorganic or organic reducing agents were able to compete seriously with these compounds in terms of efficacy, toxicological properties, price etc. The most frequently used compounds of this kind are thioglycolic acid, thiolactic acid, cysteine and cysteamine. All these thiols have in common the feature that with time, in aqueous solution, they develop a very unpleasant odor. To be accurate, there are three phases during which the odor disturbs:
1. At the time the bottle is opened when the thiol-saturated vapor space makes itself noticeable;
2. During application, when because of the large surface area of the hair, possibly also under the action of heat, a rather large amount of thiol vaporizes, and this affects the ambient air of the hairdresser salon;
3. For several days following the permanent wave treatment, particularly when the hair becomes moist (for example in fog or in a sauna): even the smallest amounts of vaporizing sulfur compounds then lead to the formation of an unpleasant “micro-climate” in the immediate vicinity of the nose.

It is known that the odor of evil-smelling compounds can be reduced by appropriate accompanying substances. Depending on the odor problem, this can be achieved via chemical effects, for example by oxidation, reduction, derivatization or the use of antioxidants as well as by physical methods, for example adsorption, dissolution or vapor pressure reduction. All substances which in any way react with the free HS− group of the waving agent necessarily deactivate it. For this reason, attempts to reduce the undesirable odor of a waving agent via the afore-indicated phases 1 and 2 with the aid of additives have thus far not achieved technical realization. In phase 3, on the other hand, the action of the waving agent takes place irreversibly so that after the end of a permanent wave treatment the use of such substances is no longer impossible.

From JP 10-45543 A is known a shampoo formulation containing the zinc salt of any desired organic acid and which is said to exert an odor-reducing effect on dyed and permanently waved hair. DE 198 57 235 A discloses aqueous, clear gels containing finely distributed metal oxides, zinc oxide among others, and which bind odors in dermatological or hair-cosmetic products. From JP 06-298626 A is known an agent based on the zinc salt of an organic acid which, obviously as a constituent of a permanent waving agent, is said to eliminate the unpleasant odor thereof without affecting wave stability. JP 54-40614 B (=JP 48-40945 A) discloses hair-conditioning agents and hair rinses containing levulinic acid and a few of its salts for the purpose of improving the smoothness and softness of hair. JP 2003-137758 A concerns odor-masking compositions for hair-treatment agents including permanent wave agents. In this publication are mentioned numerous essential oils suitable for odor combating as well as individual substances. The individual substances include among others also free levulinic acid (CAS No. 123-76-2). In JP 60-197615 and JP 60-158105 are described additional hair-treatment agents that can contain free levulinic acid.

Moreover, the odor of volatile substances given off by the human body, for example perspiration, can also be based on sulfur compounds. Thus, by the action of skin bacteria, volatile, evil-smelling sulfur compounds are set free from sulfur-containing amino acids. The compositions thus far known for combating unpleasant odors based on sulfur compounds, however, are not yet completely satisfactory. Hence, it was our goal to provide other possibilities for combating such odors.

The object of the invention is the use of
(a) at least one oxocarboxylic acid, particularly one having the general formula R—C(═O)—A—CO₂H wherein R stands for hydrogen or a monovalent organic group and A stands for a divalent organic group or for a single bond, the oxocarboxylic acid possibly being in unneutralized, partly neutralized or completely neutralized form, and
(b) at least one substance selected from among metal ions, organic bases and polymers that are substantive for keratin material
   for combating, suppressing or eliminating odors of the hair or skin, particularly the odors of sulfur-containing compounds and the odors caused by permanent wave treatment of the hair.

Oxocarboxylic acids are carboxylic acids which in addition to at least one carboxyl group as the functional group contain at least one carbonyl group, namely the aldehydo- or ketocarboxylic acids. Oxocarboxylic acids are, for example, 2-oxocarboxylic acids such as glyoxylic acid, pyruvic acid or 2-oxoglutaric acid, 3-oxocarboxylic acids, for example acetoacetic acid or 3-oxoglutaric acid, 4-oxocarboxylic acids for example levulinic acid etc. Preferred oxocarboxylic acids are the 4-oxocarboxylic acids, for example those having the general formula R—C(═O)—CH₂—CH₂—CO₂H wherein R denotes hydrogen or a monovalent organic group and preferably a C₁-C₆-alkyl group. Levulinic acid (4-oxopentanoic acid) is particularly preferred.

The metal ions are preferably ions of subgroup metals, particularly zinc ions. The polymers substantive for keratin material are preferably selected from among film-forming polymers, hair-fixing polymers and hair-care polymers. Substantive polymers are polymers having the ability to adhere to a keratin material and particularly to keratin fibers, for example hair. Suitable film-forming polymers are, in particular, those indicated in the International Cosmetic Ingredient Dictionary and Handbook, 9th edition, as having the function of “film formers”. Suitable hair-fixing polymers are in particular those indicated in the International Cosmetic Ingredient Dictionary and Handbook, 9th edition, as having the function of “hair fixatives”. Suitable hair-care polymers are, in particular, those indicated in the International Cosmetic Ingredient Dictionary and Handbook, 9th edition, as having the function of “hair conditioning agents” provided they are polymers. Examples of substantive polymers for purposes of the present invention are indicated hereinbelow.

Organic bases for the purposes of the present invention are carbon compounds with at least one group that shows a basic reaction in water, and particularly at least one primary, secondary or tertiary group. Preferred are aminoalkanols with, for example, 2 to 10 carbon atoms, for example aminomethylpropanol (AMP), triethanolamine or monoethanolamine. Particularly preferred are monoalkanolamines, especially the aminomethylpropanol 2-amino-2-methyl-1-propanol.

In a particular embodiment of the invention, the metal salts of the oxocarboxylic acids, particularly the zinc salt of levulinic acid, are not incorporated into the cosmetic product as a finished raw material, but are produced in situ. To this end, the quantities of metal salts, metal oxides, metal hydroxides or free metals wanted in the product are charged to a container first and then treated with, for example, the stoichiometrically calculated quantity of the oxocarboxylic acid. Also, it is possible to charge the oxocarboxylic acid first and then treat it with the desired quantity of metal salts, metal oxides, metal hydroxides or free metal. To this end, metallic zinc and suitable compounds thereof, particularly zinc oxide, zinc hydroxide, zinc carbonate, basic zinc carbonate or zincates etc, are preferred.

Naturally, the odor-reducing effect of leave-on products is appreciably stronger than that of rinse-off products. Hence, in leave-on products 1/10 to 1/1000 of the concentration of a compound used in a rinse-off product is sufficient to suppress or fully eliminate the permanent wave odor, for example 0.001 to 1 wt. % for leave-on products and 0.01 to 10 wt. % for rinse-off products.

Attempts to suppress the sulfur odor of permanently waved hair with appropriate raw materials have shown that free levulinic acid (4-oxopentanoic acid) is already an effective odor reducer. Surprisingly, we have found that the metal salts of levulinic acid, in particular, are capable of improving the odor of permanently waved hair for a long time if they are applied immediately or even a few days after a permanent wave treatment. To this end, the zinc salt of levulinic acid (zinc levulinate), in particular, exceeds in synergistic manner the sum of the individual effects of levulinic acid, on the one hand, and other zinc compounds, on the other. Although the soluble zinc salts of other acids also have an odor-reducing effect, the effect of zinc levulinate is much more intense than that of any other zinc compound. Based on various odor tests carried out on permanently waved hair, zinc levulinate, even when used at the lowest concentrations in rinse-off products, shows a drastic odor-reducing effect. This effect was observed after a permanent wave treatment both immediately after zinc levulinate was applied as well as up to four days later.

The odor-reducing effect of the salts of levulinic acid, particularly zinc levulinate, can be expected both when simple aqueous, alcoholic or aqueous-alcoholic solutions are used, and when such salts are used as constituents of normal product bases, such as shampoos, hair-cure compositions, hair rinses, conditioners, hair-care foams, hair tonics, ointments, styling gels, styling waxes, styling foams, hair sprays, tinting agents, colorants and perfumes. Thus, special permanent wave-deodorizing products can be produced with these substances. Furthermore, a conventional hair-cosmetic product can be provided with an additional advantage in a simple manner.

Moreover, the odor of the volatile substances given off by humans (perspiration), provided it is based on sulfur compounds, can be effectively combated with the substances presented here. Thus, the activity of skin bacteria causes sulfur-containing amino acids to release volatile, evil-smelling sulfur compounds which can be converted into an odorless form by means of the salts of the oxocarboxylic acids to be used according to the invention, particularly the zinc salt of levulinic acid. Zinc levulinate, in particular, is thus well suited for use in skin-cosmetic products such as deodorizing sticks, perfumes, deodorizing sprays, deodorizing rollers, skin creams, skin lotions, skin milks, shower products, bath additives or washing lotions.

The invention also has for an object cosmetic agents, particularly hair-treatment and skin-treatment agents, that contain a combination of
(a) at least one of the afore-described oxocarboxylic acids, that can be unneutralized, partly neutralized or completely neutralized, and
(b) at least one substance selected from among metal ions of at least one subgroup metal and polymers that are substantive for keratin material
   in a cosmetically acceptable carrier medium.

Zinc levulinate or a suitable precursor thereof is preferably used. Depending on the kind of application, the individual constituents are each used in an amount from 0.001 to 10 wt. % and preferably from 0.005 to 5 wt. %. As a rule, for leave-on products the use concentration can be one hundredth to one thousandth of that employed for rinse-off products.

Depending on the product base, the pH can be between 2.0 and 10.0 and preferably between 3.0 and 8.0. In the case of zinc levulinate, above a pH of 7.0 ammonium ions should also be present so as to keep the zinc ions in solution, otherwise the zinc ions would precipitate as the hydroxide.

The oxocarboxylates used for odor reduction can be used alone but naturally also in combination with other odor-reducing substances such as the cyclodextrins, ursolic acid etc.

Furthermore, the cosmetic agents of the invention can additionally contain at least one other hair-cosmetic active ingredient or additive. Depending on their kind and end use, the active ingredients and additives are preferably used in an amount from 0.01 to 20 wt. %, and particularly from 0.05 to 10 wt. % or from 0.1 to 5 wt. %. The active ingredients and additives can be selected from among:
hair-care agents, hair fixatives, silicone compounds, light stabilizers, oils, waxes, viscosity modifiers, preservatives, pigments, direct hair dyes, particulate substances, surfactants, oxidants, reducing agents, oxidation hair dye precursors, skin-care products, moisturizers and other deodorizing active ingredients.

The agent of the invention can be present in one of the following product forms, among others:
shampoos, hair-cure agents, hair rinses, hair conditioners, hair foams, hair tonics, ointments, for temporary shaping and/or stabilizing a hairdo (styling agents), for example hair sprays, hair coatings, fixing lotions, fixing foams, hair-styling gels, hair-styling waxes, hair-dressing creams etc, permanent, semipermanent or temporary hair colorants, for example oxidative hair colorants or nonoxidative hair tinting agents, hair-bleaching agents, permanent hair-deformation agents, for example permanent wave agents or hair-smoothing agents, perfumes, deodorizing sticks, deodorizing sprays, deodorizing rollers, deodorizing head scarfs, deodorizing creams, skin creams, skin lotions, skin milk, shower products, bath additives, washing lotions or simple aqueous, alcoholic or aqueous-alcoholic solutions.

In particular, the agent of the invention can be present
as a hair-cleaning or skin-cleaning agent together with 0.01 to 40 wt. % of at least one anionic, amphoteric or nonionic detersive surfactant and 50 to 90 wt. % of water;
as a hair spray either in combination with a propellant (aerosol spray) or in combination with a mechanical spraying device (pump spray);
as a permanent wave-fixing solution with at least one oxidant;
as an emulsion-forming hair-care agent containing water, at least one oil or solid substance and at least one emulsifier;
as hair foam containing at least one foam-forming substance and which is present in combination with a foaming device;
as a hair fixative containing at least one hair-fixing polymer;
as a hair colorant containing at least one hair dye or at least one hair dye precursor;
as a hair tonic containing at least 10 wt. % of ethanol and/or isopropanol;
as a deodorizing skin-treatment agent with at least one additional deodorizing substance.

In one embodiment, the agent of the invention contains as a hair-care or hair-fixing additive or as a substantive polymer at least one polymer with anionic or anionizable groups preferably in an amount from 0.01 to 20 wt. % or from 0.05 to 20 wt. % and particularly from 0.1 to 5 wt. %. By anionizible groups are meant acid groups, for example carboxylic acid, sulfonic acid or phosphoric acid groups, which can be deprotonated with common bases, for example organic amines or alkali metal or alkaline earth metal hydroxides. The polymers can be partly or completely neutralized with a basic neutralizing agent. Preferred are agents in which the acid groups in the polymer are neutralized to the extent of 50 to 100% and particularly of 70 to 100%. Suitable neutralizing agents are organic or inorganic bases. Examples of bases are, in particular, aminoalkanols, for example aminomethylpropanol (AMP), triethanolamine or monoethanolamine as well as ammonia, NaOH, KOH etc.

The polymer can be a homopolymer or a copolymer with acid groups-containing monomer units on a natural or synthetic basis, the monomer optionally being copolymerized with comonomers devoid of acid groups. Suitable acid groups are sulfonic acid, phosphoric acid and carboxylic acid groups among which the carboxylic acid groups are preferred. Suitable acid groups-containing monomers are, for example, acrylic acid, methacrylic acid, crotonic acid, maleic acid or maleic anhydride, maleate monoesters and particularly the C₁-C₇-alkyl esters of maleic acid, as well as the aldehydocarboxylic acids or ketocarboxylic acids. Comonomers that are not acid-substituted are, for example acrylamide, methacrylamide, alkyl- and dialkylacrylamide, alkyl- and dialkylmethacrylamide, alkyl acrylate, alkyl methacrylate, vinylcaprolactone, vinylpyrrolidone, vinyl esters, vinyl alcohol, propylene glycol or ethylene glycol, amino-substituted vinyl monomers, for example dialkylaminoalkyl acrylate, dialkylaminoalkyl methacrylate, monoalkylaminoalkyl acrylate and monoalkylaminoalkyl methacrylate, the alkyl groups of these monomers preferably being C₁-C₇-alkyl groups and particularly C₁-C₃-alkyl groups.

Suitable polymers with acid groups are, in particular, noncrosslinked homopolymers of acrylic acid or methacrylic acid or the homopolymers thereof crosslinked with polyfunctional agents, copolymers of acrylic acid or methacrylic acid with monomers selected from among acrylic acid esters or methacrylic acid esters, acrylamides, methacrylamides and vinyl-pyrrolidone, homopolymers of crotonic acid and copolymers of crotonic acid with monomers selected from among vinyl esters, acrylate or methacrylate esters, acrylamides and methacrylamides. A suitable natural polymer is, for example, shellac.

Preferred polymers with acid groups are:
the terpolymers of acrylic acid, alkyl acrylate and N-alkylacrylamide (INCI designation: acrylates/acrylamide copolymer), particularly the terpolymers of acrylic acid, ethyl acrylate and N-tert.butylacrylamide; crosslinked and uncrosslinked vinyl acetate/crotonic acid copolymers (INCI designation: VA/crotonates copolymer); copolymers of one or more C₁-C₅-alkyl acrylates, particularly C₂-C₄-alkyl acrylates, and at least one monomer selected from among acrylic acid or methacrylic acid (INCI designation: acrylates copolymer), for example the terpolymers of tert.butyl acrylate, ethyl acrylate and methacrylic acid; sodium polystyrenesulfonate; vinyl acetate/crotonic acid/vinyl alkanoate copolymers, for example the copolymers of vinyl acetate, crotonic acid and vinyl propionate; copolymers of vinyl acetate, crotonic acid and vinyl neodecanoate (INCI designations: VA/crotonates/vinyl propionate copolymer, VA/crotonates/vinyl neodecanoate copolymer); aminomethylpropanol-acrylate copolymers; copolymers of vinylpyrrolidone and at least one other monomer selected from among acrylic acid and methacrylic acid as well as, optionally, acrylate esters and methacrylate esters; copolymers of methyl vinyl ether and monoalkyl esters of maleic acid (INCI designations: ethyl ester of PVM/MA copolymer, butyl ester of PVM/MA copolymer); aminomethylpropanol salts of copolymers of allyl methacrylate and at least one other monomer selected from among acrylic acid and methacrylic acid and optionally acrylate esters and methacrylate esters; crosslinked copolymers of ethyl acrylate and methacrylic acid; copolymers of vinyl acetate, mono-n-butyl maleate and isobornyl acrylate; copolymers of two or more monomers selected from among acrylic acid and methacrylic acid and optionally acrylate esters and methacrylate esters; copolymers of octylacrylamide and at least one monomer selected from among acrylic acid and methacrylic acid and optionally acrylate esters and methacrylate esters; polyesters of diethylene glycol, cyclohexanedimethanol, isophthalic acid and sulfoisophthalic acid, the alkyl groups of the afore-said polymers as a rule preferably having 1, 2, 3 or 4 carbon atoms.

In one embodiment, the agent of the invention contains as the hair-care or hair-fixing additive or as the substantive polymer at least one zwitterionic and/or amphoteric polymer preferably in an amount from 0.01 to 20 wt. % or from 0.05 to 10 wt. % and particularly from 0.1 to 5 wt. %. Zwitterionic polymers contain simultaneously at least one anionic and at least one cationic charge. Amphoteric polymers contain at least one acid group (for example a carboxylic acid or sulfonic acid group) and at least one basic group (for example an amino group). The acid groups can be deprotonated with common bases, for example with organic amines or alkali metal or alkaline earth metal hydroxides.

Preferred zwitterionic or amphoteric polymers are:
the copolymers derived from alkylacrylamide, alkylaminoalkyl methacrylate and two or more monomers selected from among acrylic acid and methacrylic acid and optionally the esters thereof, particularly the copolymer of octylacrylamide, acrylic acid, butylaminoethyl methacrylate, methyl methacrylate and hydroxypropyl methacrylate (INCI designation: octylacrylamide/acrylates/butylaminoethyl methacrylate copolymer); copolymers derived from at least one first monomer kind containing quaternary amino groups and at least one second monomer kind containing acid groups; copolymers of fatty alcohol acrylates, alkylaminoxide methacrylate and at least one monomer selected from among acrylic acid and methacrylic acid as well as optionally acrylate esters and methacrylate esters, particularly the copolymers of lauryl acrylate, stearyl acrylate, ethylaminoxide methacrylate and at least one monomer selected from among acrylic acid and methacrylic acid and optionally the esters thereof; copolymers of methacryloylethylbetaine and at least one monomer selected from among methacrylic acid and methacrylate esters; copolymers of acrylic acid, methyl acrylate and methacrylamidopropyltrimethylammonium chloride (INCI designation: Polyquaternium-47); copolymers of acrylamidopropyltrimethylammonium chloride and acrylates or copolymers of acrylamide, acrylamidopropyltrimethylammonium chloride, 2-amidopropylacrylamide sulfonate and dimethylaminopropylamine (INCI designation: Polyquaternium-43); oligomers or polymers that can be prepared from quaternary crotonebetaines or quaternary crotonebetaine esters.

In one embodiment, the agent of the invention contains as the hair-care or hair-fixing additive or as the substantive polymer at least one cationic polymer, namely a polymer with cationic group or groups that can be converted into cationic groups, particularly primary, secondary, tertiary or quaternary amino groups preferably in an amount from 0.01 to 20 wt. % or from 0.05 to 10 wt. % and particularly from 0.1 to 5 wt. %. The cationic charge density is preferably from 1 to 7 meq/g.

The suitable cationic polymers are preferably hair-fixing or hair-conditioning polymers. Suitable polymers of component (b) preferably contain quaternary amino groups. The cationic polymers can be homopolymers or copolymers, the quaternary nitrogen groups being contained either in the polymer chain or preferably as substituents in one or more of the monomers. The monomers containing ammonium groups can be copolymerized with noncationic monomers. Suitable cationic monomers are unsaturated, free radical-polymerizable compounds bearing at least one cationic group, particularly ammonium-substituted vinyl monomers, for example trialkylmethacryloxyalkylammonium, trialkylacryloxyalkylammonium, dialkyldiallylammonium and quaternary vinylammonium monomers with cyclic, cationic nitrogen-containing groups such as pyridinium and imidazolium or quaternary pyrrolidones, for example alkylvinylimidazolium, alkylvinylpyridinium or alkylvinylpyrrolidone salts. The alkyl groups in these monomers are preferably the low alkyl groups, for example C₁-C₇-alkyl groups, and most preferably C₁-C₃-alkyl groups.

The ammonium groups-containing monomers can be copolymerized with noncationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, the alkyl groups of these monomers preferably being C₁-C₇-alkyl groups and particularly C₁-C₃-alkyl groups. Suitable polymers with quaternary amino groups are, for example, the polymers described in the CFTA Cosmetic Ingredient Dictionary under the Polyquaternium designations, for example methylvinylimidazolium chloride/vinylpyrrolidone copolymer (Polyquaternium-16) or the quaternized vinylpyrrolidone/dimethylaminoethyl methacrylate copolymer (Polyquaternium-11) as well as the quaternary silicone polymers or oligomers, for example the silicone polymers with quaternary end groups (Quaternium-80).

Preferred cationic polymers on a synthetic basis are:
poly(dimethyldiallylammonium chloride); copolymers of acrylamide and dimethyidiallylammonium chloride; quaternary ammonium polymers formed by reaction of diethyl sulfate and a copolymer of vinylpyrrolidone and dimethylaminoethyl methacrylate, particularly vinylpyrrolidone/dimethylaminoethyl methacrylate methosulfate copolymer (for example Gafquat® 755 N and Gafquat® 734); quaternary ammonium polymers of methylvinylimidazolium chloride and vinylpyrrolidone (for example LUVIQUAT® HM 550); Polyquaternium-35; Polyquaternium-57; the polymer of trimethylammoniumethyl methacrylate chloride; the terpolymers of dimethyidiallylammonium chloride, sodium acrylate and acrylamide (for example Merquat® Plus 3300); the copolymers of vinylpyrrolidone, dimethylaminopropylmethacrylamide and metyhacryloylaminopropyllauryldimethylammonium chloride; the terpolymers of vinylpyrrolidone, dimethylaminoethyl methacrylate and vinylcaprolactam (for example Gaffix® VC 713); vinylpyrrolidone/methacrylamidopropyltrimethylammonium chloride copolymers (for example Gafquat® HS 100); the copolymers of vinylpyrrolidone and dimethylaminoethyl methacrylate; the copolymers of vinylpyrrolidone, vinylcaprolactam and dimethylaminopropylacrylamide; poly- or oligoesters, derived from at least one first kind of monomer selected from a hydroxy acid substituted with at least one quaternary ammonium group, and dimethylpolysiloxanes substituted with quaternary ammonium groups at the end of the chain.

Suitable cationic polymers derived from natural polymers are, in particular, the cationic derivatives of polysaccharides, for example the cationic derivatives of cellulose, starch or guar. Also suitable are chitosan and chitosan derivatives. Cationic polysaccharides have, for example, the general formula

G—O—B—N⁺R^{a}R^{b}R^{c}X⁻

wherein
G is an anhydroglucose group, for example a starch- or cellulose anhydroglucose
B is a divalent combining group, for example alkylene, oxyalkylene, polyoxyalkylene or hydroxyalkylene;
R^{a}, R^{b} and R^{c} independently of each other stand for alkyl, aryl, alkylaryl, arylalkyl, alkoxyalkyl or alkoxyaryl, each with up to 18 carbon atoms, the total number of carbon atoms in
R^{a}, R^{b} and R^{c} preferably being at the most 20;
X is a common counterion, for example a halogen, acetate, phosphate, nitrate or alkylsulfate and preferably a chloride. Cationic celluloses are, for example, those with the INCI designation Polyquaternium-10 and Polyquaternium-24. A suitable cationic guar derivative has, for example, the INCI designation guar hydroxypropyltrimonium chloride.

Particularly preferred cationic substances are chitosan, chitosan salts and chitosan derivatives. The chitosans to be used according to the invention are fully or partly deacetylated chitins. The molecular weight can be distributed over a wide range, for example from 20,000 to about 5 million g/mol or, for example from 30,000 to 70,000 g/mol. Preferably, however, the molecular weight is above 100,000 g/mol and particularly from 200,000 to 700,000 g/mol. The degree of deacetylation is preferably from 10 to 99% and particularly from 60 to 99%. A preferred chitosan salt is chitosonium pyrrolidonecarboxylate, for example Kytamer® PC with a molecular weight of about 200,000 to 300,000 g/mol and a degree of deacetylation of 70 to 85%. Suitable chitosan derivatives are quaternized, alkylated or hydroxyalkylated derivatives, for example hydroxyethyl-, hydroxypropyl- or hydroxybutylchitosan. The chitosans or chitosan derivatives are preferably in neutralized or partly neutralized form. The degree of neutralization is preferably at least 50% and most preferably between 70 and 100%, based on the number of free base groups. In principle, all cosmetically compatible inorganic or organic acids can be used as the neutralizing agent, for example formic acid, tartaric acid, malic acid, lactic acid, citric acid, pyrrolidonecarboxylic acid, hydrochloric acid and others, among which pyrrolidonecarboxylic acid is particularly preferred.

Preferred cationic polymers on a natural basis are:
cationic cellulose derivatives of hydroxyethylcellulose and diallyldimethylammonium chloride; cationic cellulose derivatives of hydroxyethylcellulose and a trimethylammonium-substituted epoxide; chitosan and the salts thereof; hydroxyalkylchitosans and the salts thereof; alkylhydroxyalkylchitosans and the salts thereof, and N-hydroxyalkylchitosan alkyl ethers.

In another preferred embodiment, the agent of the invention contains from 0.01 to 15 wt. % and preferably from 0.5 to 10 wt. % of at least one synthetic or natural nonionic film-forming polymer. By natural polymers are meant also chemically modified polymers of natural origin. By film-forming polymers are meant polymers which when used in 0.01 to 5% aqueous, alcoholic or aqueous-alcoholic solution are capable of depositing a polymer film on the hair.

Suitable synthetic, nonionic film-forming, hair-fixing polymers are homopolymers or copolymers derived from at least one of the following monomers:
vinylpyrrolidone, vinylcaprolactam, vinyl esters, for example vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, the alkyl groups of these monomers preferably being C₁-C₇-alkyl groups and most preferably C₁-C₃-alkyl groups. Suitable are, for example, the homopolymers of vinylcaprolactam, of vinylpyrrolidone or of N-vinylformamide. Other suitable synthetic film-forming, nonionic, hair-fixing polymers are, for example, the copolymers of vinylpyrrolidone and vinyl acetate, the terpolymers of vinylpyrrolidone, vinyl acetate and vinyl propionate, polyacrylamide, polyvinyl alcohols and polyethylene glycol/polypropylene glycol copolymers. Suitable natural film-forming polymers are, for example, cellulose derivatives, for example hydroxyalkylcellulose.

Preferred nonionic polymers are:
polyvinylpyrrolidone, polyvinylcaprolactam, vinylpyrrolidone/vinyl acetate copolymers, polyvinyl alcohol, isobutylene/ethylmaleimide/hydroxyethylmaleimide copolymer and copolymers of vinylpyrrolidone, vinyl acetate and vinyl propionate.

In one embodiment, the agent of the invention contains as the hair-care additive at least one silicone compound, preferably in an amount from 0.01 to 15 wt. % and particularly from 0.1 to 5 wt. %. The silicone compounds comprise volatile and nonvolatile silicones and silicones that are soluble or insoluble in the agent. In one embodiment, the silicone is a high-molecular-weight silicone with a viscosity from 1,000 to 2,000,000 cSt and preferably from 10,000 to 1,800,000 or from 100,000 to 1,500,000 cSt at 25° C. The silicone compounds comprise polyalkyl- and polyarylsiloxanes and particularly those with methyl, ethyl, propyl, phenyl, methylphenyl and phenylmethyl groups. Preferred are polydimethylsiloxanes, polydiethylsiloxanes and polymethylphenylsiloxanes. Also preferred are the luster-imparting, arylated silicones with a refractive index of at least 1.46 or at least 1.52. In particular, the silicone compound comprise the substances with the INCI designations of cyclomethicone, dimethicone, dimethiconol, dimethicone copolyol, phenyl trimethicone, amodimethicone, trimethylsilylamodimethicone, stearyl siloxy silicate, polymethylsilsesquioxane and dimethicone crosspolymer. Also suitable are silicone resins and silicone elastomers which are highly crosslinked siloxanes. Preferred silicones are: cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide groups, polydimethylsiloxanes with terminal hydroxyl groups, phenyl-substituted polydimethylsiloxanes, silicone emulsions, silicone elastomers, silicone waxes, silicone gums, amino-substituted silicones and silicones substituted with quaternary ammonium groups.

In one embodiment, the agent of the invention contains a light stabilizer preferably in an amount from 0.01 to 10 wt. % or from 0.1 to 5 wt. % and particularly from 0.2 to 2 wt. %. In particular, the light stabilizers comprise all those mentioned in EP 11 084 696. Preferred are: 4-methoxycinnamic acid 2-ethylhexyl ester, methyl methoxycinnamate, 2-hydroxy-4-methoxybenzophenone-5-sulfonic acid and polyethoxylated p-aminobenzoates.

In one embodiment, the agent of the invention contains at least one hydrophobic oil or wax preferably in an amount from 0.01 to 20 wt. %, particularly from 0.05 to 10 wt. % and more preferably from 0.1 to 5 wt. %. The liquid, hydrophobic oils have a melting point of less than or equal to 25° C. and a boiling point of, preferably, above 250° C. and particularly above 300° C. Any oil generally known to those skilled in the art can be used to this end. Suitable are vegetable or animal oils, mineral oils (Paraffinum liquidum), silicone oils or mixture thereof. Suitable are hydrocarbon oils, for example paraffin oils or isoparaffin oils, squalane, oils from fatty acids and polyols and particularly the triglycerides.

Suitable vegetable oils are, for example, sunflower oil, coconut oil, castor oil, lanolin oil, jojoba oil, corn oil and soybean oil.

Suitable as the wax or wax-like substance is in principle any wax known from the prior art. These include animal, vegetable, mineral and synthetic waxes, microcrystalline waxes, macrocrystalline waxes, solid paraffins, petrolatum, vaselines, ozocerite, montana wax, Fischer-Tropsch waxes, polyolefin waxes, for example polybutene, beeswax, wool wax and the derivatives thereof, for example wool wax alcohols, candelilla wax, olive wax, carnauba wax, Japan wax, apple wax, hardened fats, fatty esters and fatty acid glycerides, in each case with a solidification point above 40° C., polyethylene waxes and silicone waxes. The waxes or wax-like substances have a solidification point above 40° C. and preferably above 55° C. The needle penetration number (0.1 mm, 100 g, 5 s, 25° C.; in accordance with German Industry Standard [DIN] 51 579) is preferably in the range from 2 to 70 and particularly from 3 to 40.

In one embodiment, the agent of the invention contains from 0.01 to 20 wt. %, preferably from 0.05 to 10 wt. % and particularly from 0.1 to 5 wt. % of at least one hair-conditioning additive, for example selected from among betaine, panthenol, panthenyl ethyl ether, sorbitol, protein hydrolyzates and plant extracts.

In one embodiment, the agent of the invention contains at least one viscosity modifier preferably in an amount from 0.01 to 20 wt. %, or from 0.05 to 10 wt. % and particularly from 0.1 to 5 wt. %. The viscosity-modifier is preferably a thickening polymer selected from among the copolymers of at least one first kind of monomer selected from among acrylic acid and methacrylic acid and at least one second kind of monomer selected from among the esters of acrylic acid and an ethoxylated fatty alcohol; crosslinked polyacrylic acid; crosslinked copolymers of at least one first kind of monomer selected from among acrylic acid and methacrylic acid and at least one second kind of monomer selected from among the esters of acrylic acid with C₁-C₁₀-alcohols; copolymers of at least one first kind of monomer selected from among acrylic acid and methacrylic acid and at least one second kind of monomer selected from among the esters of itaconic acid and an ethoxylated fatty alcohol; copolymers of at least one first kind of monomer selected from among acrylic acid and methacrylic acid, at least one second kind of monomer selected from among the esters of itaconic acid and an ethoxylated C₁₀-C₃₀-alcohol and a third kind of monomer selected from among C₁-C₄-aminoalkyl acrylates; copolymers of two or more monomers selected from among acrylic acid, methacrylic acid, acrylate esters and methacrylate esters; copolymers of vinylpyrrolidone and ammoniumacryloyldimethyl taurate; copolymers of ammoniumacryloyldimethyl taurate and monomers selected from among the esters of methacrylic acid and an ethoxylated fatty alcohol; hydroxyethylcellulose; hydroxypropylcellulose; hydroxypropylguar; glyceryl polyacrylate; glyceryl polymethacrylate; copolymers of at least one C₂-, C₃- or C₄-alkylene and styrene; polyurethanes; hydroxypropylstarch phosphate; polyacrylamide; copolymer of maleic anhydride and methyl vinyl ether crosslinked with decadiene; carob bean flower; guar gum; xanthane; dehydroxyxanthane; caragheenan; Karaya gum; hydrolyzed corn starch and copolymers of polyethylene oxide, fatty alcohols and saturated methylenediphenyl diisocyanate (for example PEG-150/stearyl alcohol/SMDI copolymer).

In one embodiment, the agent of the invention contains from 0.01 to 20 wt. %, preferably from 0.05 to 10 wt. % and particularly from 0.1 to 5 wt. % of at least one surfactant. The surfactant can be nonionic, anionic, cationic or zwitterionic.

Suitable nonionic surfactants are, for example:
ethoxylated fatty alcohols, fatty acids, fatty acid glycerides or alkylphenols, particularly the addition products of 2 to 30 mols of ethylene oxide and/or from 1 to 5 mols of propylene oxide to C₈-C₂₂-fatty alcohols, to C₁₂-C₂₂-fatty acids or to alkylphenols with 8 to 15 carbon atoms in the alkyl group;
C₁₂- to C₂₂-fatty acid mono- and diesters of the addition products of 1 to 30 mols of ethylene oxide to glycerol;
addition products of 5 to 60 mols of ethylene oxide to castor oil or hardened (hydrogenated) castor oil;
fatty acid sugar esters, particularly the esters of sucrose and one or two C₈-C₂₂-fatty acids, INCI: sucrose cocoate, sucrose dilaurate, sucrose distearate, sucrose laurate, sucrose myristate, sucrose oleate, sucrose palmitate, sucrose ricinoleate and sucrose stearate;
esters of sorbitan and one, two or three C8- to C₂₂-fatty acids and having a degree of ethoxylation from 4 to 20;
polyglyceryl fatty acid esters, particularly from one, two or more C₈- to C₂₂-fatty
acids and polyglycerol, preferably with 2 to 20 glyceryl units;
alkyl glucosides, alkyloligoglucosides and alkylpolyglucosides with C₈- to C₂₂-alkyl groups, for example decyl glucoside or lauryl glucoside.

Suitable anionic surfactants are, for example, the salts and esters of carboxylic acids, alkyl ether sulfates and alkyl sulfates, fatty alcohol ether sulfates, sulfonic acid and the salts thereof (for example sulfosuccinates or fatty acid isethionates), phosphate esters and the salts thereof and acylamino acids and the salts thereof. Detailed descriptions of these anionic surfactants can be found in the publication “FIEDLER—Lexikom der Hilfstoffe” [FIEDLER—Encyclopedia of Auxiliary Substances], vol. 1, fifth edition (2002), pages 97 to 102, the disclosures of which are hereby specifically incorporated by reference. Preferred surfactants are the mono- di- and/or triesters of phosphoric acid and the addition products of 2 to 30 mols of ethylene oxide to C₈- to C₂₂-fatty alcohols.

Suitable amphoteric surfactants are, for example, the derivatives of aliphatic quaternary ammonium, phosphonium and sulfonium compounds having the formula

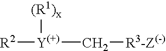

wherein R denotes a straight-chain or branched alkyl, alkenyl or hydroxyalkyl group with 8 to 18 carbon atoms and from 0 to about 10 ethylene oxide units and from 0 to 1 glycerol unit; Y denotes an N-, P- or S-containing group; R denotes an alkyl or monohydroxyalkyl group with 1 to 3 carbon atoms; X equals 1 when Y is a sulfur atom and X equals 2 when Y is a nitrogen atom or phosphorus atom; R³ denotes an alkylene or hydroxyalkylene group with 1 to 4 carbon atoms, and Z⁽⁻⁾ denotes a carboxylate, sulfate, phosphonate or phosphate group.

Other amphoteric surfactants such as the betaines are also suitable. Examples of betaines include C₈-C₁₈-alkylbetaines such as cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis-(2-hydroxypropyl)alphacarboxyethylbetaine; C₈-C₁₈-sulfobetaines such as cocodimethylsulfopropylbetaine, stearyldimethylsulfopropylbetaine, lauryldimethylsulfoethylbetaine and laurylbis(2-hydroxyethyl)sulfopropylbetaine; the carboxyl derivatives of imidazole, the C₈-C₁₈-alkyldimethylammonium acetates, the C₈-C₁₈-alkyldimethylcarbonylmethylammonium acetates, the C₈-C₁₈-alkyldimethylcarbonylmethylammonium salts as well as the C₈-C₁₈-fatty acid alkylamidobetaines, for example coco fatty acid amidopropylbetaine and N-cocofatty acid amidoethyl-N-[2-(carboxymethoxy)ethyl]glycerol (CTFA name: cocoamphocarboxyglycinate).

Suitable cationic surfactants contain amino groups or quaternized hydrophilic ammonium groups which in solution bear a positive charge and can be represented by the general formula

N^{(+)R}¹R²R³R⁴X⁽⁻⁾

wherein R¹ to R⁴ independently of each other denote aliphatic groups, aromatic groups, alkoxy groups, polyoxyalkylene groups, alkylamido groups, hydroxyalkyl groups, aryl groups or alkaryl groups with 1 to 22 carbon atoms, in which at least one group has at least 6 and preferably at least 8 carbon atoms and X⁻ stands for an anion, for example a halogen, acetate, phosphate, nitrate or alkylsulfate and preferably a chloride. Besides the carbon atoms and hydrogen atoms, the aliphatic groups can additionally contain crosslinks or other groups, for example other amino groups.

Examples of suitable cationic surfactants are the chlorides or bromides of alkyldimethylbenzylammonium salts, alkyltrimethylammonium salts, for example cetyltrimethylammonium chloride or bromide, tetradecyltrimethylammonium chloride or bromide, alkyldimethylhydroxyethylammonium chloride or bromide, the dialkyldimethylammonium chlorides or bromides, alkylpyridinium salts, for example lauryl- or cetylpyridinium chloride, alkylamidoethyltrimethylammonium ether sulfate and compounds with a cationic character such as amine oxides, for example alkylmethylamine oxide or alkylaminoethyidimethylamine oxide. Particularly preferred are C₈-C₂₂-alkyldimethylbenzylammonium compounds, C₈-C₂₂-alkyltrimethylammonium compounds, particularly cetyltrimethylammonium chloride, C₈-C₂₂-alkyldimethylhydroxyethylammonium compounds, di-(C₈-C₂₂-alkyl)-dimethylammonium compounds, C₈-C₂₂-alkylpyridinium salts, C₈-C₂₂-alkylamidoethyltrimethylammonium ether sulfates, C₈-C₂₂-alkylmethylamine oxides and C₈-C₂₂-alkylaminoethyidimethylamine oxides.

In one embodiment, the agent of the invention contains from 0.01 to 5 wt. % and particularly from 0.05 to 1 wt. % of at least one preservative. Suitable preservatives are the substances listed in the International Cosmetic Ingredient Dictionary and Handbook, 9th edition, under “Preservatives”, for example phenoxyethanol, benzylparaben, butylparaben, ethylparaben, isobutylparaben, isopropylparaben, methylparaben, propylparaben, iodopropinylbutyl carbamate, methyldibromoglutaronitrile, DMDM and hydantoin.

A particular embodiment concerns a hair-care agent. Hair-care agents are conditioners, treatments, hair cure products, rinses and the like. The hair-care agent contains at least one active hair-care ingredient selected from among the afore-said silicone compounds, cationic or amino-substituted surfactants and cationic or amino-substituted polymers. The active hair-care ingredient can be used in an amount from 0.01 to 10.0 wt. % and particularly from 0.01 to 5.0 wt. %, based on the end product. After application to dry, moist or wet hair, the hair-care agent of the invention can either remain on the hair or after an appropriate contact time it can be rinsed out. The contact times depend on the kind of hair. As a general guideline, one starts with a contact time between 0.5 and 30 minutes, particularly between 0.5 and 10 minutes and preferably between 1 and 5 minutes.

Other suitable cationic or amino-substituted surfactants besides the afore-said cationic surfactants are those having the formula R1—NH—(CH₂)ₙ—NR2R3 or formula R1—NH—(CH₂)ₙ—N⁺R2R3R4 X⁻ wherein R1 denotes an acyl or alkyl group with 8 to 24 carbon atoms which can be branched or unbranched, saturated or unsaturated, the acyl group and/or alkyl group possibly containing one or more OH groups, R2, R3 and R4 independently of each other denote hydrogen, alkyl groups or alkoxyalkyl groups with 1 to 6 carbon atoms which can be equal or different, saturated or unsaturated and possibly substituted with one or more hydroxyl groups, X⁻ is an anion, particularly a halide ion or a compound of general formula RSO₃⁻ wherein R stands for a saturated or unsaturated alkyl group with 1 to 4 carbon atoms and n is an integer from 1 and 10 and preferably from 2 to 5.

The hair-care agent is preferably an amido amine and/or a quaternized amido amine of the afore-indicated formulas wherein R1 denotes a branched or unbranched, saturated or unsaturated acyl group with 8 to 24 carbon atoms that can contain at least one OH group. Also preferred are amines and/or quaternized amines wherein at least one of the R2, R3 and R4 groups denotes a group of general formula CH₂CH₂OR5, wherein R5 stands for an alkyl group with 1 to 4 carbon atoms, hydroxyethyl or H. Suitable amines or amido-amines that can optionally be quaternized are in particular those with the INCI designations ricinoleamidopropylbetaine, ricinoleamidopropyldimethylamine, ricinoleamidopropyldimethyl lactate, ricinoleamidopropylethyldimonium ethosulfate, ricinoleamidopropyltrimonium chloride, ricinoleamidopropyltrimonium methosulfate, cocamidopropylbetaine, cocamidopropyldimethylamine, cocamidopropylethyldimonium ethosulfate, cocamidopropyltrimonium chloride, behenamidopropyidimethylamine, isostearylamidopropyldimethylamine, stearylamidopropyldimethylamine, Quaternium-33 and undecyleneamidopropyltrimonium methosulfate.

In another embodiment, the agent of the invention contains at least one pigment. The pigments can be colored so as to impart color effects to the product composition or to the hair, or the pigments can be luster pigments which impart a luster effect to the product composition or to the hair. The color effects or luster effects on the hair are preferably temporary, namely they last up to the next hair washing and can be removed by washing the hair with a common shampoo. In the product composition, the pigments are present in an undissolved form and in an amount from 0.01 to 25 wt. % and preferably from 5 to 15 wt. %. The preferred particle size is from 1 to 200 μm, particularly from 3 to 150 μm and most preferably from 10 to 100 μm. The pigments are practically insoluble colorants in the application medium and can be inorganic or organic. Mixed inorganic-organic pigments are also possible. Inorganic pigments are preferred. The advantage of inorganic pigments is their outstanding light, weather and heat resistance. The inorganic pigments can be of natural origin, for example prepared from chalk, ocher, umber, green earth, burned Terra di Siena or graphite. The pigments can be white, for example titanium dioxide or zinc dioxide, they can be black, for example black iron oxide, or they can be colored pigments, for example ultramarine or red iron oxide or luster-imparting pigments, metal-effect pigments, nacreous pigments or fluorescent or phosphorescent pigments, and preferably at least one pigment being a colored, non-white pigment. Suitable are metal oxides, hydroxides and oxide hydrates, mixed-phase pigments, sulfur-containing silicates, metal sulfides, complex metal cyanides, metal sulfates, chromates and molybdates as well metals as such (bronze pigments). Particularly well suited are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510) and carmine (cochineal).

Particularly preferred are nacreous and colored pigments based on mica and coated with a metal oxide or a metal oxychloride such as titanium dioxide or bismuth oxychloride as well as optionally other color-imparting substances such as iron oxides, Prussian blue, ultramarine, carmine etc. and the color of which can be adjusted by varying the coating thickness. Such pigments are marketed by Merck, Germany, under the tradenames Rona®, Colorona®, Dichrona® and Timiron®.

Organic pigments are, for example, the natural pigments sepia, gamboge, bone charcoal, Cassel brown, indigo, chlorophyll and other vegetable pigments. Synthetic organic pigments are, for example, the azo pigments, anthraquinoids, indigoids, dioxazine-, quinacridone-, phthalocyanine-, isoindolinone-, perylene- and perinone pigments, metal complex pigments, alkali blue pigments and diketopyrrolopyrrol pigments.

In one embodiment, the agent of the invention contains at least one other particulate substance different from component (a) in an amount from 0.01 to 10 wt. % and particularly from 0.05 to 5 wt. %. Suitable substances are, for example, those that are solid at room temperature (25° C.) and are in the form of particles. Suitable are silica, silicates, aluminates, clays, mica, salts, particularly inorganic metal salts, metal oxides, for example titanium dioxide, minerals and polymer particles. In the agent, the particles are present in undissolved, preferably stably dispersed form and after application to the hair and evaporation of the solvent can deposit themselves on the hair in solid form. A stable dispersion can be obtained by providing the composition with a flow limit that is high enough to prevent the solid particles from settling. A sufficient flow limit can be obtained by use of an adequate amount of a suitable gel former. Preferred particulate substances are silica (silica gel, silicon dioxide) and metal salts, particularly inorganic metal salts, silica being particularly preferred. Metal salts are, for example, alkali metal or alkaline earth metal halides such as sodium chloride or potassium chloride and alkali metal or alkaline earth metal sulfates, such as sodium sulfate or magnesium sulfate.

One other embodiment concerns a kit for lasting hair deformation which contains a first composition containing one of the afore-said combinations of oxocarboxylic acid and metal ions or substantive polymers, to be used according to the invention, and at least one second composition selected from among compositions that contain at least one reducing agent (permanent wave agent), particularly a keratin-reducing mercapto compound, preferably in an amount from 0.5 to 15 wt. %, and compositions containing at least one oxidant, for example hydrogen peroxide (fixation). A preferred kit contains in addition to the first composition also a reducing agent-containing permanent wave agent composition as well as an oxidant-containing fixing composition. The permanent wave agent is preferably in the form of an aqueous, alkaline (pH=5 to 10) composition which contains as the keratin-reducing mercapto compound, for example, cysteine, cysteamine, N-acetyl-L-cysteine, a mercaptocarboxylic acid, for example thioglycolic acid or thiolactic acid, or a salt of a mercaptocarboxylic acid, for example an ammonium or guanidine salt of thioglycolic acid or thiolactic acid. The alkalinity is adjusted to the required level by addition of ammonia, organic amines, ammonium- or alkali metal carbonates or hydrogen carbonates. Also feasible is a neutral or acidic hair-deformation agent (pH =4.5 to 7) which in an aqueous medium has an effective content of sulfites or mercaptocarboxylate esters. Preferably used in the first case are sodium- or ammonium sulfite or a salt of sulfurous acid with an organic amine, for example monoethanolamine and guanidine, at a concentration of about 2 to 12 wt. % (calculated as SO₂). Particularly suitable in the second case are thioglycolic acid monoglycol esters or glycerol esters at a concentration of about 5 to 50 wt. % (corresponding to a free thioglycolic acid content of 2 to 16 wt. %).

The agent of the invention for lasting hair deformation can also contain a mixture of the afore-said keratin-reducing compounds. After a contact time sufficient for lasting hair deformation, the hair is rinsed with water and then oxidatively post-treated (fixed). The fixative used for the oxidative post-treatment can contain at least one oxidant and one oxocarboxylate metal salt of the invention, or any desired fixative previously used for such treatment can be used. Examples of oxidants that can be contained in such a fixative are sodium- and potassium bromate, sodium perborate, urea peroxide and hydrogen peroxide. The oxidant concentration can range from about 0.5 to 10 wt. %.

Both the permanent wave agent and the fixative of the invention can be in the form of an aqueous solution or emulsion as well as in thickened form on an aqueous basis, particularly a cream, gel or paste. It is also possible to fill aerosol cans with this agent under pressure and dispense it as a foam.

Another embodiment relates to a hair colorant. The colorant can be either an oxidative or a nonoxidative colorant based on oxidation and/or direct dyes that in themselves are known. The total amount of the oxidation dye precursors present in the agent of the invention is preferably about 0.01 to 12 wt. % and particularly 0.2 to 6 wt. %. Suitable oxidation dye precursors are, for example, the following developers and couplers and self-coupling compounds.

The total amount of direct dyes in the agent of the invention is from about 0.01 to 7 wt. % and preferably from about 0.2 to 4 wt. %. Suitable direct dyes which in themselves are known to those skilled in the art are, for example, the triphenylmethane dyes, aromatic nitro dyes, azo dyes, quinone dyes, cationic or anionic dyes.

The oxidant composition used can be a composition of the invention containing at least one oxidant and metal salts of oxocarboxylic acids or any desired composition which until now has been used for such treatment. Suitable oxidants used for color development are primarily hydrogen peroxide or the compounds of addition thereof to urea, melamine or sodium borate in the form of a 1 to 12% and preferably 1.5 to 6% aqueous solution.

The agent of the invention can, in addition, contain other common additives for hair-treatment agents, for example perfume oils, opacifying agents, for example ethylene glycol distearate, styrene/PVP copolymers or a polystyrene; moisturizers; luster-imparting agents; product-tinting agents and antioxidants, each preferably in an amount from 0.01 to 10 wt. %, the total amount not exceeding 10 wt. %.

The agent of the invention can be in any form that is suitable for hair-treatment agents and skin-treatment agents, for example in the form of a solution, emulsion, dispersion, cream or gel. The agent can also be sprayed or foamed in admixture with a gaseous propellant or by means of a mechanically operated spraying device. The agent of the invention is preferably packaged in an aqueous, alcoholic or aqueous-alcoholic medium with preferably at least 10 wt. % of water. Suitable alcohols are, in particular, the lower alcohols with 1 to 4 carbon atoms usually employed for cosmetic purposes, for example ethanol and isopropanol. The pH of the agent of the invention can be in the range from 2 to 10. Particularly preferred is the pH range between 3 and 8, unless special forms of application require a different pH. Suitable as additional co-solvents are organic solvents or a mixture of solvents with a boiling point below 400° C., in an amount from 0.1 to 15 wt. % and preferably from 1 to 10 wt. %. Particularly suitable as additional co-solvents are unbranched or branched hydrocarbons such as pentane, hexane, isopentane and cyclic hydrocarbons such as cyclopentane and cyclohexane. Other, particularly preferred water-soluble solvents are glycerol, ethylene glycol and propylene glycol in an amount of up to 30 wt. %.

In one embodiment, the agent of the invention is in the form of a gel, in the form of a viscous lotion or in the form of a spray gel which is sprayed with a mechanical device, and contains at least one of the afore-said thickening polymers preferably in an amount of 0.05 to 10 wt. % and particularly 0.1 to 2 wt. % and has a viscosity of at least 250 mPa.s (determined with a Bohlin Rheometer CS, spindle C25, at 25° C. at a shearing rate of 50 s⁻¹). The viscosity of the gel is preferably from 500 to 50,000 mPa.s and particularly from 1,000 to 15,000 mPa.s at 25° C.

In one embodiment, the agent of the invention is in the form of an O/W emulsion, a W/O emulsion or a microemulsion and contains at least one of the afore-said oils or waxes emulsified in water and at least one of the afore-said surfactants.

In one embodiment, the agent of the invention is in the form of a spray product, either in combination with a mechanical pump spray device or in combination with a propellant selected from among propane, butane, dimethyl ether and fluorinated hydrocarbons. In particular, the spray products can be free of metal ions and contain a combination of oxocarboxylic acid or a salt thereof not derived from metals, with at least one substantive polymer. In addition, an aerosol spray preferably contains from 15 to 85 wt. % and particularly from 25 to 75 wt. % of a propellant and is packaged in a pressure container. Suitable propellants are, for example, the lower alkanes, for example n-butane, isobutane and propane, or mixtures thereof as well as dimethyl ether or fluorocarbons such as F 152a (1,1-difluroethane) or F 134 (tetrafluoroethane) as well as the gaseous propellants suitable for use at the pressures involved, for example N₂, N₂O and CO₂.

A non-aerosol hair spray is sprayed with the aid of a suitable mechanically operated spraying device. By mechanically operated spraying devices are meant devices which spray a composition without the use of a propellant. A suitable mechanical spraying device is, for example, a spraying pump or an elastic container provided with a spray valve, and in which the cosmetic agent of the invention is packaged under pressure. Under these conditions the elastic container expands, and when the spray valve is opened the agent is continuously released as a result of the contraction of the elastic container.

In one embodiment, the agent of the invention is in the form of a foamable (effervescent) product in combination with a foaming device and contains at least one common foam-forming substance known for this purpose, for example at least one foam-forming surfactant or at least one foam-forming polymer. By devices for foaming are meant devices that make possible the foaming of a liquid with or without the use of a propellant. A suitable mechanical foaming device is, for example, a commercial foaming pump or an aerosol foam head. The product is used either in combination with a mechanical foaming pump device (pump foam) or in combination with at least one propellant (aerosol foam) and is preferably present in an amount from 1 to 20 wt. % and particularly from 2 to 10 wt. %. The propellants are selected, for example, from among propane, butane, dimethyl ether and fluorinated hydrocarbons. The agent is foamed just before application and in the form of a foam worked into the hair, after which it can be rinsed out or left in the hair without being rinsed out.

In one embodiment, the agent of the invention is in the form of a hair wax, namely it has a waxy consistency and contains at least one of the afore-said waxes preferably in an amount from 0.5 to 30 wt. % and optionally other water-insoluble substances. The waxy consistency is preferably characterized in that the needle penetration number (measuring unit 0.1 mm, test weight 100 g, test duration 5 seconds, test temperature 25° C., according to German Industry Standard [DIN] 51 579) is higher than or equal to 10 and most preferably higher than or equal to 20, and that the solidification point of the product is higher than or equal to 30° C. and lower than or equal to 70° C. and most preferably in the range from 40 to 55° C. Suitable waxes and water-insoluble substances are, in particular, emulsifiers with an HLB value below 7, silicone oils, silicone waxes, waxes (for example wax alcohols, wax acids, wax esters and, in particular, natural waxes such as beeswax, carnauba wax etc.), fatty alcohols, fatty acids, fatty esters or hydrophilic waxes, for example high-molecular-weight polyethylene glycols with a molecular weight from 800 to 20,000 and preferably from 2,000 to 10,000 g/mol.

When the hair-treatment agent of the invention is in the form of a hair lotion, it is essentially a non-viscous or slightly viscous fluid solution, dispersion or emulsion containing at least 10 wt. % and preferably from 20 to 95 wt. % of a cosmetically compatible alcohol. Suitable alcohols are, in particular, the alcohols with 1 to 4 carbon atoms commonly used for cosmetic purposes, for example ethanol and isopropanol.

When the agent of the invention is in the form of a hair cream, it is preferably an emulsion and contains additionally either viscosity-imparting constituents in an amount from 0.1 to 10 wt. %, or the required viscosity and creamy consistency is created in the usual manner by micelle formation with the aid of suitable emulsifiers, fatty acids, fatty alcohols, waxes etc.

When the hair-treatment agent is in the form of a hair-cleaning agent it contains additionally at least one detersive surfactant, preferably from 0.01 to 25 wt. % and particularly from 5 to 20 wt. %, of at least one anionic, amphoteric and/or nonionic surfactant and from 50 to 90 wt. % of water. The hair-cleaning agent preferably has a pH from 3 to 8 and particularly from 4 to 7. Suitable surfactants are, for example, the afore-said ones. Preferred surfactants for a hair-cleaning agent of the invention are selected from among alkali metal or alkaline earth metal salts of C₁₀-C₁₈-alkylsulfates, C₁₀-C₁₈-alkylsulfonates, C₁₀-C₁₈-alkylbenzenesulfonates, C₁₀-C₁₈-xylenesulfonates and C₁₀-C₁₈-alkyl ether sulfates ethoxylated with 1 to 10 ethylene oxide units; the ethoxylated half esters of sulfosuccinic acid of general formula R¹(OCH₂CH₂)ₘ—O₂C—CH₂CH(SO₃M)—CO₂M, wherein R¹ denotes a C₁₀-C₁₈-alkyl group, M denotes an alkali metal cation or alkaline earth metal cation, and m stands for an integer from 1 to 10; the alkyl ether carboxylates having the formula R²(CH₂CH₂)ₙ—OCH₂COOM, wherein R denotes a C₁₀-C₁₈-alkyl group, M denotes an alkali metal cation or alkaline earth metal cation and n stands for an integer from 1 to 20; ethoxylated fatty alcohols with 12 to 18 carbon atoms, polyglycerol ethers of saturated or unsaturated fatty alcohols and alkylphenols with 8 to 30 carbon atoms in the alkyl group and 1 to 10 glyceryl groups in the molecule; fatty acid alkanolamides; ethoxylated sorbitan fatty acid esters, C₁₀-C₁₈-alkylpolyglucosides, C₁₀-C₁₈-alkylbetaines and amphoteric surfactants having the formula

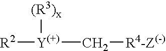

wherein R² denotes a straight-chain or branched alkyl-, alkenyl- or hydroxyalkyl group with 8 to 18 carbon atoms and from 0 to 10 ethylene oxide units and from 0 to 1 glycerol unit; Y denotes an N-, P- or S-atom; R³ denotes an alkyl or monohydroxyalkyl group with 1 to 3 carbon atoms; x equals 1 when Y is a sulfur atom and x equals 2 when Y is a nitrogen or phosphorus atom; R⁴ denotes an alkylene- or hydroxyalkylene group with 1 to 4 carbon atoms, and Z stands for a carboxylate, sulfate, phosphonate or phosphate group.

The invention also has for an object a method for hair treatment whereby
the hair is subjected to a treatment for lasting hair shape alteration and
before that the hair is treated simultaneously or subsequently with a combination of
(a) at least one oxocarboxylic acid, particularly one having the general formula R—C(═O)—A—CO₂H wherein R stands for hydrogen or a monovalent organic group and A stands for a divalent organic group or a single bond, the oxocarboxylic acid possibly being in unneutralized, partly neutralized or completely neutralized form, and
(b) at least one substance selected from among metal ions and polymers that are substantive for keratin material.

Following the shape-altering treatment, the hair is preferably treated with a post-treatment agent containing a combination of (a) levulinic acid or a neutralized form thereof and (b) zinc ions after which the post-treatment agent is rinsed out (rinse product) or remains on the hair without being rinsed out (leave-on product).

The subject matter of the invention will now be explained in greater detail by way of the following examples.

### EXAMPLES

### Preparation of the Zinc Salts

Unless the required zinc salts are commercially available, they are prepared by dissolving zinc oxide (ZnO) in the acid in question. To this end 0.2 mol of a monobasic acid is added to 0.1 mol of ZnO (8.1 g) in the form of 10% aqueous suspension, and the mixture is stirred to complete dissolution. At this point, the pH is about 7. Any possibly undissolved residual zinc oxide is filtered off, and the filtrate is evaporated to dryness. The material remaining as residue is then used directly. In the case of polybasic acids, their molarities are correspondingly divided by two or three.

### Preparation of the Hair Strands

The test substances were tested on hair that had been freshly permanently waved. To this end, hair braids were cut into 2.5-cm pieces, bleached for 30 minutes and washed with perfume-free shampoo. To obtain fully odorless strands, the strands were then dipped three times into a mixture of isopropanol 25%/ethanol 25%/water 50%. The hair was kept for 24 hours in a conditioning room (20° C. at 85% relative humidity) and then cut to a weight of 2 g. This was followed by an odor test. Ten strands per post-treatment agent to be tested were wound onto rollers (diameter: 13 mm) and kept overnight in the conditioning room (20° C. at 85% relative humidity). The next morning, these strands were subjected to permanent waving.

### Permanent Wave Treatment

Ten rollers holding 2 g of hair each were placed in a crystallizing dish and covered twice with 20 g of a commercial wave lotion, the rollers being turned over after the first 20 g to ensure uniform distribution. The rollers were then placed in a drier at 40° C. for 15 min, after which they were rinsed with water for 5 minutes namely for 2 min under running city water and for an additional 3 min by immersing them in water. The hair was then dabbed dry with a paper napkin. Afterward, the fixation was carried out with the aid of a commercial foam fixative. To this end, the rollers were immersed for 3 min in 500 mL of fixing solution. Excess fixing solution was then poured off, and the rollers were allowed to stand for an additional 7 minutes. They were then again rinsed with water for 5 min (see above) and dabbed dry with a paper napkin. Finally, the hair was detached from the roller and placed in a drier at 40° C. for 60 minutes.

### Tests Performed on Hair Strands (Leave-on Product)

To test the efficacy of the compounds as permanent wave post-treatment agents, the zinc salts and the corresponding zinc-free acids alone were incorporated at two different pH values into common cosmetic product bases. Zinc citrate (trizinc dicitrate) made soluble with a slight excess of citric acid was tested as the reference substance for a zinc salt without the odor-influencing counterion. The oxocarboxylic acids were used both as the sodium salts and in the form of their zinc salts. The pH values were then adjusted with citric acid or NaOH.

In the following example, 0.5 mL of a leave-on conditioner was massaged into the freshly permanently waved hair (2 g in each case), and after a contact time of 10 min the odor was evaluated by five test subjects (Table 1). In each case, the odor evaluation was performed on 3 test strands. The average values of the evaluation results are tabulated.

Whereas levulinic acid showed some odor-reducing action in strong acids, the performance of the zinc ions was best in a less strongly acidic medium. None of the components tested, however, reached the efficacy of zinc levulinate which was unique in the entire pH range tested.

### Rating Scale

Each symbol stands for one test strand.
◯=odor indistinguishable from that of blank
+=odor better than that of blank
++=odor appreciably better than that of blank/odorless

TABLE 1
Odor of Permanently Waved Hair Strands by Leave-on Method
(Base: Conditioner)

| Test | | | | | | | | |
|---|---|---|---|---|---|---|---|---|
| Substance | Day 1²⁾ | Day 1²⁾ | Day 2³⁾ | Day 2³⁾ | Day 3²⁾ | Day 3²⁾ | Day 4³⁾ | Day 4³⁾ |
| (0.01 wt. %) | pH 2-3 | pH 4-5 | pH 2-3 | pH 4-5 | pH 2-3 | pH 4-5 | pH 2-3 | pH 4-5 |
| Blank¹⁾ | ∘ ∘ ∘ | ∘ ∘ ∘ | ∘ ∘ ∘ | ∘ ∘ ∘ | ∘ ∘ ∘ | ∘ ∘ ∘ | ∘ ∘ ∘ | ∘ ∘ ∘ |
| Trizinc | + ∘ ∘ | ∘ + + | ∘ + ∘ | ∘ + + | ∘ ∘ + | ∘ + + | ∘ + ∘ | + + + |
| dicitrate | | | | | | | | |
| Na glyoxylate | ∘ + + | ∘ + ∘ | + + ∘ | + + ∘ | ∘ ∘ + | ∘ + + | ∘ + + | + ∘ + |
| Zn glyoxylate | ∘ + + | + ∘ ∘ | ∘ ∘ ∘ | ∘ + + | ∘ ∘ + | + ∘ + | ∘ ∘ + | + + ∘ |
| Na levulinate | + + + | + ∘ + | + + + | + ∘ + | ∘ + + | + + ∘ | + + + | + + + |
| Zn levulinate | + + + | ++ ++ | + + + | + + ++ | + + + | ++ ++ | + + + | + ++ |
| | | ++ | | | | ++ | | ++ |
| Na pyruvate | + ∘ + | ∘ ∘ + | ∘ + ∘ | ∘ + ∘ | ∘ + + | ∘ + + | ∘ + + | + + ∘ |
| Zn pyruvate | ∘ + ∘ | + + + | ∘ ∘ ∘ | + + + | ∘ ∘ ∘ | ∘ + + | ∘ ∘ ∘ | + + + |

¹⁾Citric acid;
²⁾wet;
³⁾dry

### Tests Performed on Hair Strands (Rinse-off Product)

Following is an example of evaluations carried out with a rinse-off product. The method was the same as previously described hereinabove with the difference that after a 5-min contact time the product was rinsed off with city water. The initial odor ratings were then obtained. A hair cure agent was used as the base. Because of the shorter contact time, the concentration of the active ingredient was increased by a factor of 100, namely to 0.1 wt. %.

TABLE 2
Odor of Permanently Waved Hair Strands by Rinse-off Method
(Base: Hair-cure Agent)

| Test | | | | | | | | |
|---|---|---|---|---|---|---|---|---|
| Substance | Day 1²⁾ | Day 1²⁾ | Day 2³⁾ | Day 2³⁾ | Day 3²⁾ | Day 3²⁾ | Day 4³⁾ | Day 4³⁾ |
| (0.01 wt. %) | pH 2-3 | pH 4-5 | pH 2-3 | pH 4-5 | pH 2-3 | pH 4-5 | pH 2-3 | pH 4-5 |
| Blank¹⁾ | ∘ ∘ ∘ | ∘ ∘ ∘ | ∘ ∘ ∘ | ∘ ∘ ∘ | ∘ ∘ ∘ | ∘ ∘ ∘ | ∘ ∘ ∘ | ∘ ∘ ∘ |
| Trizinc | ∘ ∘ ∘ | ∘ ∘ + | + ∘ ∘ | ∘ + + | ∘ ∘ + | ∘ + + | ∘ ∘ + | ∘ + + |
| dicitrate | | | | | | | | |
| Na glyoxylate | ∘ ∘ ∘ | ∘ ∘ ∘ | + ∘ + | + ∘ + | ∘ ∘ + | + + ∘ | ∘ + + | ∘ + + |
| Zn glyoxylate | ∘ ∘ ∘ | ∘ + + | ∘ ∘ ∘ | + ∘ + | ∘ ∘ + | + + ∘ | ∘ ∘ + | ∘ + + |
| Na levulinate | + + + | + ∘ + | + + ∘ | + ∘ ∘ | ∘ + + | + ∘ ∘ | ∘ ∘ + | + ∘ ∘ |
| Zn levulinate | + + + | ++ ++ | + + + | + + + | + + + | + + + | + + + | ++ + ++ |
| | | ++ | | | | | | |
| Na pyruvate | ∘∘ + | ∘ + + | ∘ + + | ∘ + + | ∘ + + | ∘ + + | ∘ + + | ∘ + + |
| Zn pyruvate | ∘ ∘ ∘ | ∘ + ∘ | ∘ ∘ ∘ | + ∘ + | ∘ ∘ ∘ | ∘ + + | ∘ ∘ ∘ | + + + |

¹⁾Citric acid;
²⁾wet;
³⁾dry

### FORMULATION EXAMPLES

### (Amounts in wt. %)

### Example 1

### Permanent Wave Post-Treatment Shampoo

| | 1A | 1B | 1C |
|---|---|---|---|
| Sodium lauryl ether sulfate (25%) | 40.0 | 40.0 | 40.0 |
| NaCl | 4.0 | 4.0 | 4.0 |
| Zinc levulinate | 0.2 | 2.0 | 5.0 |
| Water | to 100 | to 100 | to 100 |

### Example 2

### Hair Shampoo for Intensive Protection Against Permanent Wave Odor

35.0 of sodium lauryl ether sulfate (25%)
4.0 of Tego Betain® L7 (cocamidopropylbetaine, 30%)
1.5 of NaCl
4.0 triethanolamine
0.1 of formic acid
0.1 of benzoic acid
0.1 of perfume oil
8.0 zinc levulinate
to 100.0 water

### Example 3

### Hair Spray Against Permanent Wave Odor

| | 3A | 3B | 3C |
|---|---|---|---|
| Vinyl acetate/crotonic acid copolymer | 2.000 | 2.000 | 2.000 |
| 2-Amino-2-methyl-1-propanol | 0.160 | 0.160 | 0.16 |
| Ethanol | 37.840 | 37.840 | 37.840 |
| Levulinic acid | 0.001 | 0.005 | 0.01 |
| Zinc chloride | — | 0.003 | 0.005 |
| Perfume oil | 0.100 | 0.100 | 0.10 |
| Propane/butane | to 100 | to 100 | to 100 |

### Example 4

### Cream Shampoo for Permanently Waved Hair

| | 4A | 4B | 4C | 4D |
|---|---|---|---|---|
| Sodium lauryl ether sulfate, 27% | 35.0 | 35.0 | 35.0 | 35.0 |
| Stearic acid | 9.0 | 9.0 | 9.0 | 9.0 |
| Zinc searate | 0.1 | 0.5 | 1.0 | 2.0 |
| Sodium levulinate | 1.0 | 3.0 | 6.0 | 10.0 |
| NaCl | 3.0 | 3.0 | 3.0 | 3.0 |
| Triethanolamine, pure | 4.0 | 4.0 | 4.0 | 4.0 |
| Propyl p-hydroxybenzoate | 0.1 | 0.1 | 0.1 | 0.1 |
| Water | to 100 | to 100 | to 100 | to 100 |

### Example 5

### Permanent Wave Fixing Solution with Odor Suppression

| | 5A | 5B | 5C |
|---|---|---|---|
| Hydrogen peroxide | 4.6 | 4.6 | 4.6 |
| Citric acid | 0.2 | 0.2 | 0.2 |
| Levulinic acid | 0.5 | 1.0 | 1.5 |
| Zinc oxide | 0.1 | 0.3 | 0.5 |
| Perfume oil | 0.1 | 0.1 | 0.1 |
| Water | to 100 | to 100 | to 100 |

### Example 6

### Intensive Hair-care Agent with Deodorizing Effect

| | 6A | 6B | 6C |
|---|---|---|---|
| Glycerol monostearate, neutral | 6.0 | 6.0 | 6.0 |
| Lanolin alkoxylate | 2.0 | 2.0 | 2.0 |
| Levulinic acid | 1.0 | 5.0 | 9.0 |
| Zinc oxide | 0.3 | 1.5 | 3.0 |
| Cetyl alcohol | 2.0 | 2.0 | 2.0 |
| Mixture of lanolin alcohol and | 1.0 | 1.0 | 1.0 |
| paraffin oil | | | |
| Tris-(oligooxyethyl)alkylammonium | 1.5 | 1.5 | 1.5 |
| phosphate | | | |
| Hydroxyethylcellulose | 0.2 | 0.2 | 0.2 |
| Citric acid | 0.1 | 0.1 | 0.1 |
| Sorbic acid | 0.2 | 0.2 | 0.2 |
| Water | to 100.0 | to 100.0 | to 100.0 |

### Example 7

### Hair-care Agent for Permanently Waved Hair

| | 7A | 7B |
|---|---|---|
| Glycerol monostearate | 6.0 | 6.0 |
| Lanolin alkoxylate | 2.0 | 2.0 |
| Cetyl alcohol | 2.0 | 2.0 |
| Mixture of lanolin alcohol and paraffin oil | 1.0 | 1.0 |
| Tris-(oligooxyethyl)alkylammonium phosphate | 1.5 | 1.5 |
| Hydroxyethylcellulose | 0.2 | 0.2 |
| Citric acid | 0.1 | 0.1 |
| Sorbic acid | 0.1 | 0.1 |
| Perfume oil | 0.1 | 0.1 |
| Zinc levulinate | 0.5 | 1.5 |
| Water | to 100 | to 100 |

### Example 8

### Foam Conditioner for Protection Against Sulfur Odor

| | 8A | 8B |
|---|---|---|
| PVP/vinylimidazolium methochloride copolymer | 5.00 | 5.00 |
| PVP/PVA copolymer | 1.00 | 1.00 |
| Polyoxyethylene-12-cetylstearyl alcohol | 0.15 | 0.15 |
| Perfume oil | 0.10 | 0.10 |
| Zinc acetate | 0.0005 | 0.005 |
| Levulinic acid | 0.001 | 0.01 |
| Propane/butane | 10.00 | 10.00 |
| Water | to 100 | to 100 |

### Example 9

### O/W Hair Dressing Cream with Deodorizing Function

| | 9A | 9B | 9C | 9D | 9E | 9F |
|---|---|---|---|---|---|---|
| Alkyl ether | 3.0 | 3.0 | 3.0 | 3.0 | 3.0 | 3.0 |
| phosphate | | | | | | |
| Polyacrylic | 1.2 | 1.2 | 1.2 | 1.2 | 1.2 | 1.2 |
| acid (Carbo- | | | | | | |
| pol ® 940) | | | | | | |
| Paraffin oil | 17.0 | 17.0 | 17.0 | 17.0 | 17.0 | 17.0 |
| Perfume oil | 0.3 | 0.3 | 0.3 | 0.3 | 0.3 | 0.3 |
| Triethanol- | 1.5 | 1.5 | 1.5 | 1.5 | 1.5 | 1.5 |
| amine | | | | | | |
| Preservative | 0.5 | 0.5 | 0.5 | 0.5 | 0.5 | 0.5 |
| Levulinic | 0.3 | 0.3 | 0.3 | 0.3 | 0.3 | 0.3 |
| acid | | | | | | |
| NaOH | 0.1 | — | — | — | — | — |
| KOH | — | 0.15 | — | — | — | — |
| Ammonia solu- | — | — | 0.17 | — | — | — |
| tion, 26% | | | | | | |
| Calcium | — | — | — | 0.1 | — | — |
| hydroxide | | | | | | |
| Magnesium | — | — | — | — | 0.5 | — |
| chloride | | | | | | |
| Zinc hydrox- | — | — | — | — | — | 0.15 |
| ide, basic | | | | | | |
| Water | to 100 | to 100 | to 100 | to 100 | to 100 | to 100 |

### Example 10

### Hair Fixative for Long, Permanently Waved Hair

| | 10A | 10B |
|---|---|---|
| Vinylpyrrolidone/vinyl acetate | 6.0 | 6.0 |
| copolymer (Luviskol ® VA 55E) | | |
| Perfume oil | 0.3 | 0.3 |
| Ethanol, 96% | 40.0 | 40.0 |
| Zinc levulinate | 0.002 | 0.02 |
| Water | to 100 | to 100 |

### Example 11

### Tinting Foam for Permanently Waved Hair

| | 11A | 11B |
|---|---|---|
| Ethanol, 96% | 10.0 | 10.0 |
| Polyquaternium-11 | 7.5 | 7.5 |
| Direct hair dye | 0.2 | 0.2 |
| Silicone oil | 0.2 | 0.2 |
| Perfume oil | 0.3 | 0.3 |
| Zinc oxide | 1.0 | 0.5 |
| Levulinic acid | 3.0 | 1.5 |
| Water | to 100 | to 100 |
| Gaseous propellant | 10.0 | 10.0 |

### Example 12

### Cream Hair Colorant for Permanently Waved Hair

| | 12A | 12B |
|---|---|---|
| Stearyl alcohol | 8.00 | 8.00 |
| Paraffin oil | 13.00 | 13.00 |
| Wool grease | 6.00 | 6.00 |
| Perfume | 0.30 | 0.30 |
| p-Toluenediamine | 0.70 | 0.70 |
| Resorcinol | 0.05 | 0.05 |
| Aminophenol | 0.06 | 0.06 |
| EDTA | 0.20 | 0.20 |
| Ammonia (25%) | 2.00 | 2.00 |
| Sodium sulfite | 1.00 | 1.00 |
| Zinc levulinate | 1.00 | 5.00 |
| Water | to 100 | to 100 |

### Example 13

### Hair Tonic for Permanently Waved Hair

| | 13A | 13B |
|---|---|---|
| Ethanol, 96% | 50 | 50 |
| Isopropanol | 10 | 10 |
| Perfume oil | 0.15 | 0.15 |
| Panthenol | 0.20 | 0.20 |
| Polyvinylpyrrolidone; Luviskol ® K 30 | 0.05 | 0.05 |
| Salicylic acid | 0.10 | 0.10 |
| Menthol | 0.02 | 0.02 |
| Camphor | 0.01 | 0.01 |
| Allantoin | 0.10 | 0.10 |
| Zinc levulinate | 0.1 | 0.5 |
| Water | to 100 | to 100 |

### Example 14

### Antiperspirant Deodorizing Emulsion

| | 14A | 14B |
|---|---|---|
| Hydagen ® deodorant ¹⁾ | 1.5 | 1.5 |
| Glyceryl monostearate | 5.0 | 5.0 |
| Cetyl alcohol | 3.0 | 3.0 |
| Ceteareth-12 | 1.5 | 1.5 |
| Ceteareth-20 | 1.5 | 1.5 |
| Cetyl oleate | 2.5 | 2.5 |
| Aluminum hydrochloride, 50%, Locron ® L | 10.0 | 10.0 |
| Ethyl p-hydroxybenzoate | 0.3 | 0.3 |
| Zinc levulinate | 0.1 | 0.5 |
| Perfume oil | 0.3 | 0.3 |
| Water | to 100 | to 100 |

¹⁾ Triethyl citrate:2,6-ditert.butyl-p-cresol 90:10

### Example 15

### Deodorizing Washing Lotion

| | 15A | 15B |
|---|---|---|
| Sodium lauryl ether sulfate (28%) | 40.0 | 40.0 |
| Cocobetaine (30%), Dehyton ® AB 30 | 5.0 | 5.0 |
| Hydagen ® deodorant ¹⁾ | 1.5 | 1.5 |
| Elfacos ® GT 282 L ²⁾ | 5.0 | 5.0 |
| Levulinic acid | 2.0 | 4.0 |
| Zinc powder, fine | 0.1 | 0.2 |
| Perfume oil | 0.5 | 0.5 |
| Water | to 100 | to 100 |

¹⁾ Triethyl citrate:2,6-ditert.butyl-p-cresol 90:10
²⁾ Hydrogenated Talloweth-60 myristyl glycol

1. Use of a combination of
(a) at least one oxocarboxylic acid, said acid possibly being unneutralized, partly neutralized or completely neutralized, and
(b) at least one substance selected from among metal ions, organic bases and polymers that are substantive for keratin material, to combat, suppress or eliminate odors of the hair or skin.

2. Use as defined in claim 1, for combating, suppressing or eliminating odors caused by permanent wave treatment of hair.

3. Use as defined in claim 1, characterized in that the oxocarboxylic acid has the general formula R—C(═O)—A—CO₂H wherein R stands for hydrogen or a monovalent organic group and A stands for a divalent organic group or for a single bond.

4. Use as defined in claim 1, characterized in that the oxocarboxylic acid is a 4-oxocarboxylic acid.

5. Use as defined in claim 1, characterized in that the oxocarboxylic acid is levulinic acid (4-oxopentanoic acid).

6. Use as defined in claim 1, characterized in that the metal ions are ions of subgroup metals.

7. Use as defined in claim 1, characterized in that a combination of (a) levulinic acid or a neutralized form thereof and (b) zinc ions is used.

8. Use as defined in claim 1, characterized in that the combination of oxocarboxylic acid and metal salt is used either directly in the form of the metal salt of the oxocarboxylic acid or the metal salt is formed in situ from the oxocarboxylic acid and a substance selected from among metal salts, metal oxides, metal hydroxides and the free metal.

9. Use as defined in claim 1, characterized in that the polymers substantive for keratin material are selected from among film-forming polymers, hair-fixing polymers and hair-care polymers and/or that the organic bases are selected from among monoalkanolamines.

10. Method for hair treatment whereby
by use of a sulfur-containing reducing agent the hair is subjected to a permanently hair-shape altering treatment and
before, simultaneously or subsequently, the hair is treated with a combination of
(a) at least one oxocarboxylic acid, said oxocarboxylic acid possibly being unneutralized, partly neutralized or completely neutralized, and
(b) at least one substance selected from among metal ions, organic bases and polymers substantive for keratin material.

11. Method as defined in claim 10, characterized in that after the shape-altering treatment the hair is treated with a post-treatment agent containing a combination of (a) levulinic acid or a neutralized form thereof and (b) zinc ions, and that the post-treatment agent is then rinsed out (rinse product) or left on the hair without being rinsed out (leave-on product).

12. Cosmetic agent containing a combination of
(a) at least one oxocarboxylic acid, said oxocarboxylic acid possibly being unneutralized, partly neutralized or completely neutralized, and
(b) at least one substance selected from among metal ions of at least one subgroup metal, monoalkanolamines and polymers substantive for keratin material in a cosmetically acceptable carrier medium.

13. Agent as defined in claim 12, characterized in that it contains zinc levulinate.

14. Agent as defined in claim 1, characterized in that it contains the combination of oxocarboxylic acid and metal ion in an amount from 0.001 to 10 wt. %.

15. Agent as defined in claim 1, characterized in that additionally it contains at least one hair-cosmetic or skin-cosmetic active ingredient or additive.

16. Agent as defined in claim 15, characterized in that it contains the hair-cosmetic or skin-cosmetic active ingredient or additive in an amount from 0.01 to 20 wt. %.

17. Agent as defined in claim 1, characterized in that it is in one the following product forms: shampoos, hair-cure agents, hair rinses, hair conditioners, hair-care foams, hair tonics, ointments, hair styling gels, hair styling waxes, hair styling foams, hair sprays, hair tinting agents, hair colorants, perfumes, deodorizing sticks, deodorizing sprays, deodorizing rollers, deodorizing head scarfs, deodorizing creams, skin creams, skin lotions, skin milk, shower products, bath additives, washing lotions or simple aqueous, alcoholic or aqueous-alcoholic solutions.

18. Agent as defined in claim 1, characterized in that it is in one of the following product forms:
as a hair- or skin-cleaning agent containing from 0.01 to 40 wt. % of at least one anionic, amphoteric or nonionic detersive surfactant and from 50 to 90 wt. % of water;
as a hair spray in combination with either a propellant (aerosol spray) or in combination with a mechanical spraying device (pump spray);
as a permanent wave fixative solution containing at least one oxidant;
as an emulsified hair-care agent and water containing at least one oil material or fatty material and at least one emulsifier;
as a hair foam and containing at least one foam-generating substance and being in combination with a foaming device;
as a hair-fixing agent and containing at least one hair-fixing polymer;
as a hair colorant and containing at least one hair dye or at least one hair dye precursor;
as a hair tonic containing at least 10 wt. % of ethanol and/or isopropanol as a deodorizing skin treatment agent and containing at least one additional deodorizing active ingredient.

19. Kit containing a first composition containing a combination of
(a) at least one oxocarboxylic acid, said oxocarboxylic acid possibly being unneutralized, partly neutralized or completely neutralized, and
(b) at least one substance selected from among metal ions and polymers substantive for keratin material
and at least one second composition selected from among:
compositions containing at least one reducing agent (permanent wave agent) and
compositions containing at least one oxidant (permanent wave fixative).
